(19) United States
(12) Patent Application Publication
(10) Pub. No.: US 2009/0229618 A1
(43) Pub. Date: Sep. 17, 2009

(54) SLEEP POSITIONING APPARATUS

(75) Inventors: James P. Sotelo, JR., Kings Mountain, NC (US); Karen M. Sotelo, Kings Mountain, NC (US)

Correspondence Address: ADAMS INTELLECTUAL PROPERTY LAW, P.A., Suite 2350 Charlotte Plaza, 201 South College Street, CHARLOTTE, NC, 28244, US

(73) Assignee: SERENITY AND PROSPERITY ENTERPRISES, LLC, Kings Mountain, NC (US)

(21) Appl. No.: 12/047,436

(22) Filed: Mar. 13, 2008

(51) Int. Cl. A61F 5/56; A61F 5/37; A61B 19/00

(52) U.S. Cl. 128/848; 5/494; 128/873; 128/869

(57) ABSTRACT

A sleep positioning apparatus for restricting the movement of a person wearing a garment from a desired sleeping position to an undesired sleeping position, comprising an interchangeable resiliently deformable roll inhibiting element having openings there through; and a fastener assembly for connecting the roll inhibiting element to the garment of a person, wherein the fastener assembly comprises an elongated band with a clip on one end and a separable ring on the opposite end, the clip for connecting with the garment of a person and the separable ring for connecting within the openings of the roll inhibiting element, thereby allowing interchangeability of the roll inhibiting element, and the clip and the garment forming a substantially flexible joint, thereby maintaining the roll inhibiting element in substantially the same orientation and position relative to the person, when the person is in the desired sleeping position.

### TECHNICAL FIELD AND BACKGROUND OF THE INVENTION

Many attempts have been made to create an effective sleep positioning device. Historically, these devices have failed for a variety of reasons including being impractical due to weight, cost, comfort and efficacy issues.

One particular problem that previous sleep positioning devices have attempted to address is the problem of snoring. Snoring is heavily associated with sleeping position. Snoring is a common problem with many people and appears to be more problematic as one ages. Studies have shown between 30% and 50% of people snore and snoring is known to cause sleep deprivation for the user and anyone else near the snorer. In addition, snoring has been shown to cause daytime drowsiness, lack of focus and even some psychological issues.

One manner in which to reduce snoring is to position the person in a manner where the air passages remain relatively unblocked thus allowing relatively unimpeded breathing during a person's sleep. For example, when a person sleeps on their back, the muscles in their tongue relax and the tongue is pulled back towards the throat. This causes the air passage to become blocked by the tongue and the person then begins to snore. The best way to prevent this snoring is to reposition the person into a desired sleeping position in order to keep their air passage relatively clear.

It is well known in the art to place a pillow or similar object behind one's back in order to deter the person from rolling over onto their back and then blocking their air passage. This solution is undesirable for a variety of reasons. Multiple pillows would have to be placed around the entire exterior because a person could change positions in a multitude of directions. The user could inadvertently push away the pillow thereby eliminating the effectiveness. Similar devices such as wedges or blocks positioned on a belt worn by the person are bulky, or lack versatility, comfort, aesthetics and the like.

Therefore it is desirable in the art to provide a sleep positioning device that has acceptable cost, efficacy, comfort and adaptability to the user.

### SUMMARY OF THE INVENTION

In an embodiment, a sleep positioning apparatus is provided for restricting the movement of a person wearing a garment from a desired sleeping position to an undesired sleeping position, including an interchangeable resiliently deformable roll inhibiting element having openings there through; and a fastener assembly for connecting the roll inhibiting element to the garment of a person, wherein the fastener assembly includes an elongated band with a clip on one end and a separable ring on the opposite end, the clip for connecting with the garment of a person and the separable ring for connecting within the openings of the roll inhibiting element, thereby allowing interchangeability of the roll inhibiting element, and the clip and the garment forming a substantially flexible joint, thereby maintaining the roll inhibiting element in substantially the same orientation and position relative to the person, when the person is in the desired sleeping position.

In another embodiment of the present invention, a sleep positioning apparatus for restricting the movement of a person wearing a garment from a desired sleeping position to an undesired sleeping position, includes a resiliently deformable, generally spherical roll inhibiting element; and a fastener assembly for connecting the roll inhibiting element to the garment of a person, wherein the fastener assembly includes an elongated band with a clip on one end and an attachment on the opposite end for connecting with the roll inhibiting element, the clip and the garment forming a substantially flexible joint, thereby maintaining the roll inhibiting element in substantially the same orientation and position relative to the person.

According to another embodiment, a sleep positioning apparatus for restricting the movement of a person wearing a garment from a desired sleeping position to an undesired sleeping position includes an interchangeable resiliently deformable roll inhibiting element having openings there through, and a fastener assembly for connecting the roll inhibiting element to the garment of a person, wherein the fastener assembly includes an elongated band with a clip on one end and a separable ring on the opposite end, the clip for connecting with the garment of a person and the separable ring for connecting with the openings of the roll inhibiting element, thereby allowing interchangeability of the roll inhibiting element with another roll inhibiting element of a different size or shape.

In another embodiment, the band defines a length direction and a width direction and is adjustable in length.

In yet another embodiment, the roll inhibiting element is generally spherical.

According to another embodiment, the roll inhibiting element is generally hollow.

### BRIEF DESCRIPTION OF THE DRAWINGS

Some of the objects of the invention have been set forth above. Other objects and advantages of the invention will appear as the description of the invention proceeds when taken in conjunction with the following drawings, in which:

FIG. 1 is a perspective view of a sleep positioning device according to the present invention;

FIG. 2 is a rear view of person connected with the sleep positioning device; and

FIG. 3 is a side view of a person showing the person moving towards an undesired sleeping position.

### DESCRIPTION OF THE PREFERRED EMBODIMENTS AND BEST MODE

In the following description, like reference characters designate like or corresponding parts throughout the several views. Also in the following description, it is to be understood that such terms as “forward,” “rearward,” “left,” “right,” “upwardly,” “downwardly,” and the like are words of convenience and are not to be construed as limiting terms.

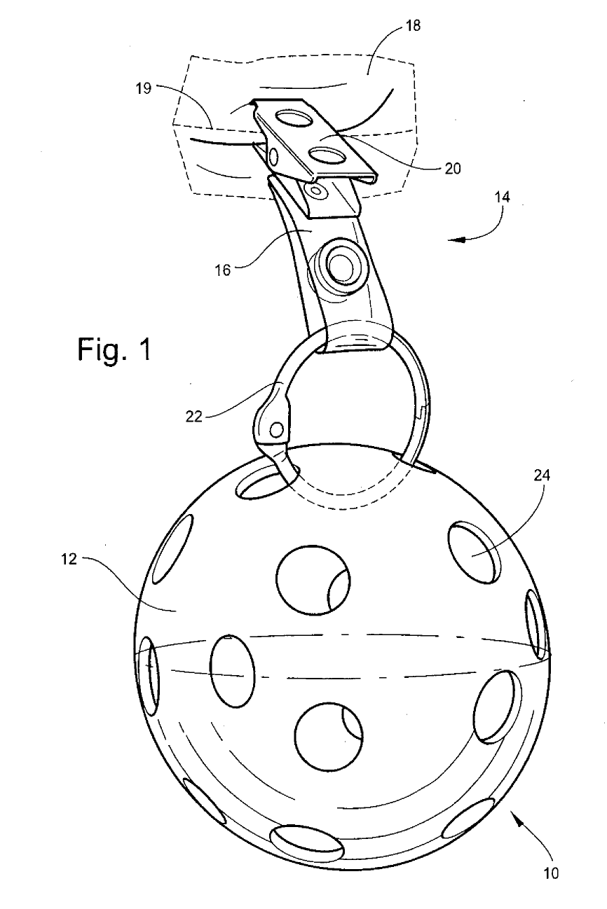

Referring now to the drawings in general and FIG. 1 in particular, it will be understood that the illustrations are for the purpose of describing a preferred embodiment of the invention and are not intended to limit the invention thereto. As shown in FIG. 1, a sleep positioning device, generally designated 10 is constructed according to the present invention. The sleep positioning device 10 includes a roll inhibiting element 12 connected with a fastening assembly, generally designated 14. The fastening assembly 14 includes a separable ring 22 at one end of a band 16 and a clip 20 at the opposite end of the band 16. The separable ring 22 may be connected with the roll inhibiting element 12 through openings 24 in the roll inhibiting element 12. The clip 20 is configured to be fastened to the garment 18 of a person. It is readily appreciable that the garment is typically of a flexible material and thereby provides a flexible joint 19 at the connection of the clip 20 to the garment 18.

The roll inhibiting element 12 may be a generally spherical shape. The roll inhibiting element 12 may also be resiliently deformable, thereby allowing the roll inhibiting element 12 to collapse under the weight of a person and return to its original form once the weight is removed. The roll inhibiting element 12 may also be replaced with another roll inhibiting element if the user desires a different element for shape, size or softness reasons. Also, the band is adjustable in length.

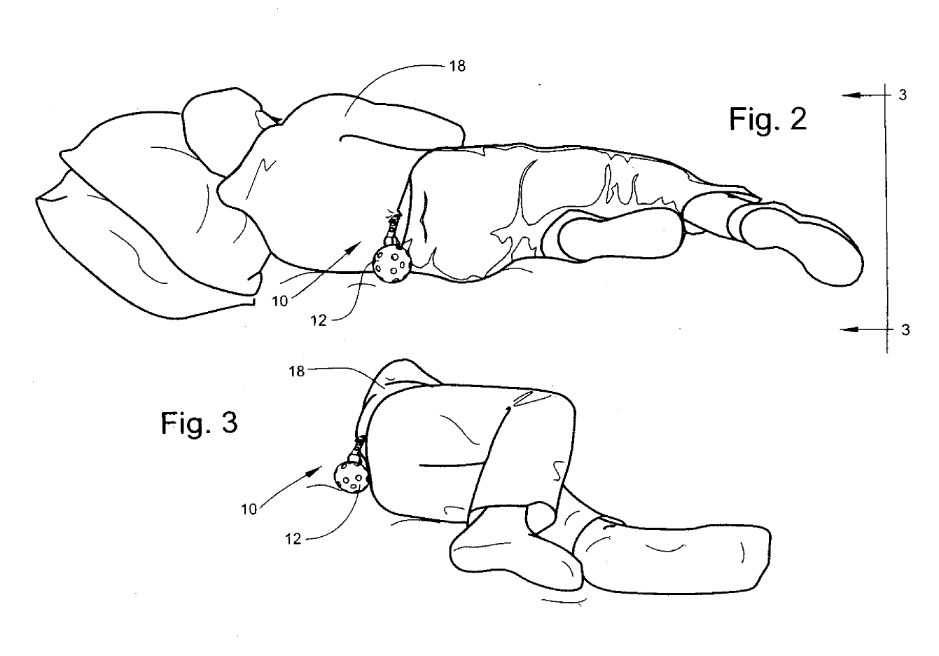

As seen in FIG. 2, the sleep positioning device 10 is connected with the person's garment 18. As should be readily apparent, the sleep positioning device 10 will maintain substantially the same orientation and position relative to the person.

As shown in FIG. 3, the sleep positioning device 10 is connected with the person's garment 18. When the person begins to change from a desired sleeping position to an undesired sleeping position, the roll inhibiting element 12 lies between the person and a surface. Upon contacting the roll inhibiting element, the user experiences discomfort and returns to the desired sleeping position.

Certain modifications and improvements will occur to those skilled in the art upon a reading of the foregoing description. For example, the fastening assembly could be a belt running through the openings of the roll inhibiting element. The roll inhibiting element could also be provided in a variety of shapes and still follow the purpose of the claims as set forth. Furthermore, while sections of this application have implied that sleeping on one's back may be undesirable, there are instances where a frontal or even side laying position may be undesirable. For example, sleeping face down is associated with an increase in sudden infant death syndrome and acne problems. It should be understood that all such modifications and improvements have been deleted herein for the sake of conciseness and readability but are properly within the scope of the following claims.

We claim:

1. A sleep positioning apparatus for restricting the movement of a person wearing a garment from a desired sleeping position to an undesired sleeping position, comprising:
(a) a resiliently deformable, generally spherical roll inhibiting element; and
(b) a fastener assembly for connecting the roll inhibiting element to the garment of a person, wherein the fastener assembly comprises an elongated band with a clip on one end and an attachment on the opposite end for connecting with the roll inhibiting element, the clip and the garment forming a substantially flexible joint, thereby maintaining the roll inhibiting element in substantially the same orientation and position relative to the person.

2. The sleep positioning apparatus according to claim 1, wherein the band defines a length direction and a width direction and is adjustable in length.

3. The sleep positioning apparatus according to claim 1, wherein the roll inhibiting element is generally spherical.

4. The sleep positioning apparatus according to claim 1, wherein the roll inhibiting element is generally hollow.

5. A sleep positioning apparatus for restricting the movement of a person wearing a garment from a desired sleeping position to an undesired sleeping position, comprising:
(a) an interchangeable resiliently deformable roll inhibiting element having openings there through; and
(b) a fastener assembly for connecting the roll inhibiting element to the garment of a person, wherein the fastener assembly comprises an elongated band with a clip on one end and a separable ring on the opposite end, the clip for connecting with the garment of a person and the separable ring for connecting with the openings of the roll inhibiting element, thereby allowing interchangeability of the roll inhibiting element with another roll inhibiting element of a different size or shape.

6. The sleep positioning apparatus according to claim 5, wherein the roll inhibiting element is generally spherical.

7. The sleep positioning apparatus according to claim 5, wherein the roll inhibiting element is generally hollow.

8. A sleep positioning apparatus for restricting the movement of a person wearing a garment from a desired sleeping position to an undesired sleeping position, comprising:
(a) an interchangeable resiliently deformable roll inhibiting element having openings there through; and
(b) a fastener assembly for connecting the roll inhibiting element to the garment of a person, wherein the fastener assembly comprises an elongated band with a clip on one end and a separable ring on the opposite end, the clip for connecting with the garment of a person and the separable ring for connecting within the openings of the roll inhibiting element, thereby allowing interchangeability of the roll inhibiting element, and the clip and the garment forming a substantially flexible joint, thereby maintaining the roll inhibiting element in substantially the same orientation and position relative to the person, when the person is in the desired sleeping position.

9. The sleep positioning apparatus according to claim 8, wherein the roll inhibiting element is generally spherical.

10. The sleep positioning apparatus according to claim 8, wherein the inhibiting element is generally hollow.

11. The sleep positioning apparatus according to claim 8, wherein the band defines a length direction and a width direction and is adjustable in length.
